(19) United States
(12) Patent Application Publication
(10) Pub. No.: US 2006/0042645 A1
(43) Pub. Date: Mar. 2, 2006

(54) Hair coloring strips and method of using same

(76) Inventors: David Watson, Fripp Island, SC (US); John Thomas Leaptrott III, Fripp Island, SC (US)

Correspondence Address: B. Craig Killough;Barnwell Whaley Patterson & Helms, LLC, P.O. Drawer H, Charleston, SC, 29402-0197, US

(21) Appl. No.: 10/928,068

(22) Filed: Aug. 27, 2004

(51) Int. Cl. A61K 8/18

(52) U.S. Cl. 132/208; 132/222

(57) ABSTRACT

A hair coloring strip having an appendage that extends to an exterior of the coloring strip. The appendage is affixed to the coloring strip so that, when the appendage is pulled by manual pressure, the hair coloring strip is released from the hair of the client for easy removal.

### FIELD OF THE INVENTION

This invention relates to hairstyling generally, and is more particularly directed to a device and method that is useful in hair coloring.

### BACKGROUND OF THE INVENTION

Hair stylists use coloring strips as accessories to color highlighting. Coloring strips are commonly formed of aluminum foil or plastic sheets, although they may be formed from other similar sheet like materials, such as paper. Coloring strips formed from any suitable material may be referred to herein as “foils,” even though the coloring strip is not formed of aluminum or other metal foil.

In use, the coloring strip is placed underneath several selected hair follicles. The end of the coloring strip is positioned as closely as possible to the scalp. The coloring material is applied to the selected hair follicles. A highlight board may be used as support. The coloring strip is then folded in half.

Many stylists prefer coloring strips that will remain closed upon folding, and which are non-absorbent. Metals foils and certain plastics meet these requirements. Some coloring strips are designed to stick to themselves upon folding.

Hair coloring chemicals and materials are time sensitive. That is, the hair coloring material is designed to be left in the hair for a preferred period of time. It is important for the hairstylist to be able to quickly and efficiently remove the coloring strips, and the attendant coloring material, since numerous coloring strips may be used for a single client. A goal of the present invention is to provide a device and method of hair coloring using hair coloring strips, wherein the coloring strips are easy to install, and easy to remove, while also providing for efficient hair coloring.

### SUMMARY OF THE INVENTION

The present invention is a hair coloring strip having an appendage that extends to an exterior of the coloring strip. The appendage is affixed to the coloring strip so that, when the appendage is pulled by manual pressure, the hair coloring strip is released from the hair of the client for easy removal.

### DESCRIPTION OF THE DRAWINGS

FIG. 1 shows a first embodiment of the hair coloring strip of the present invention.

FIG. 2 shows a second embodiment of the hair coloring strip of the present invention.

FIG. 3a shows the hair coloring strip on the present invention in position in a client's hair.

FIG. 3b shows the hair coloring strip of the present invention in position in the client's hair, with the appendage in position for removal.

FIG. 4a shows the hair coloring strip on the present invention in position in a client's hair.

FIG. 4b shows the hair coloring strip of the present invention in position in the client's hair, with the appendage in position for removal.

### DESCRIPTION OF THE PREFERRED EMBODIMENTS

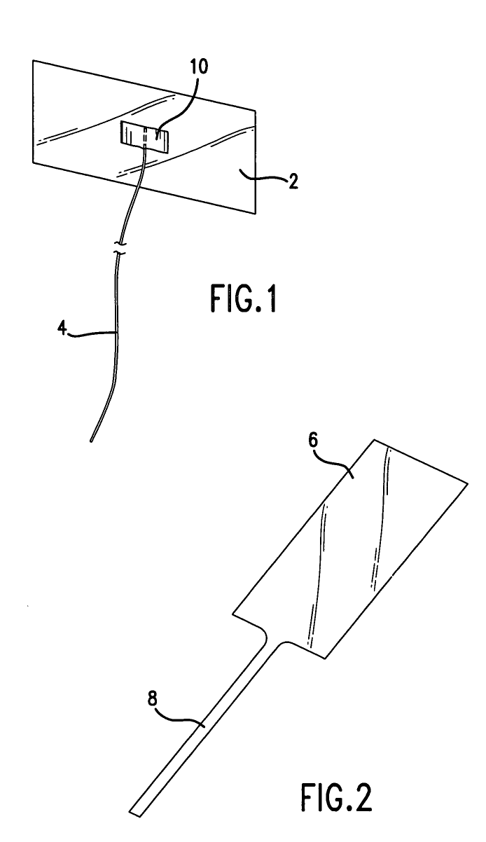

The term “appendage” is used herein to indicate a smaller elongated member that projects from a larger body, which is the coloring strip. As shown in FIG. 1, the coloring strip 2 has a smaller, but longer, appendage that extends from the coloring strip. The appendage is in the form of a string 4. In FIG. 2, the coloring strip 6 has an appendage in the form of a smaller elongated tail 8 that extends from the larger coloring strip.

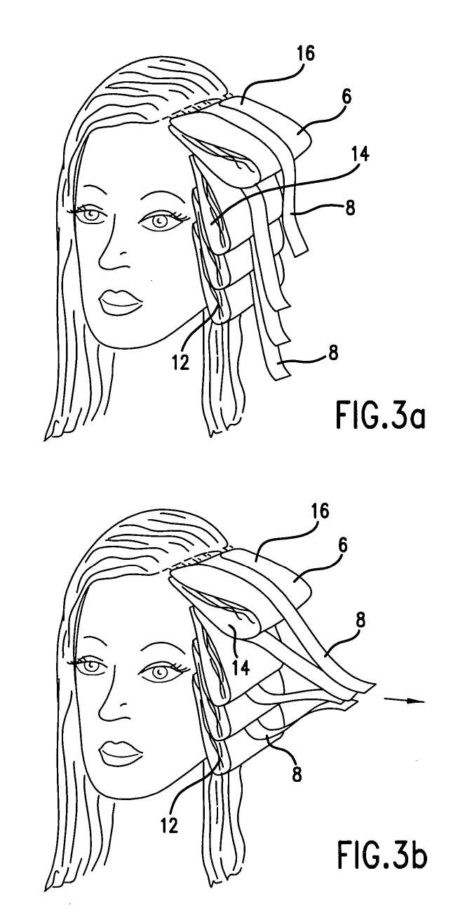

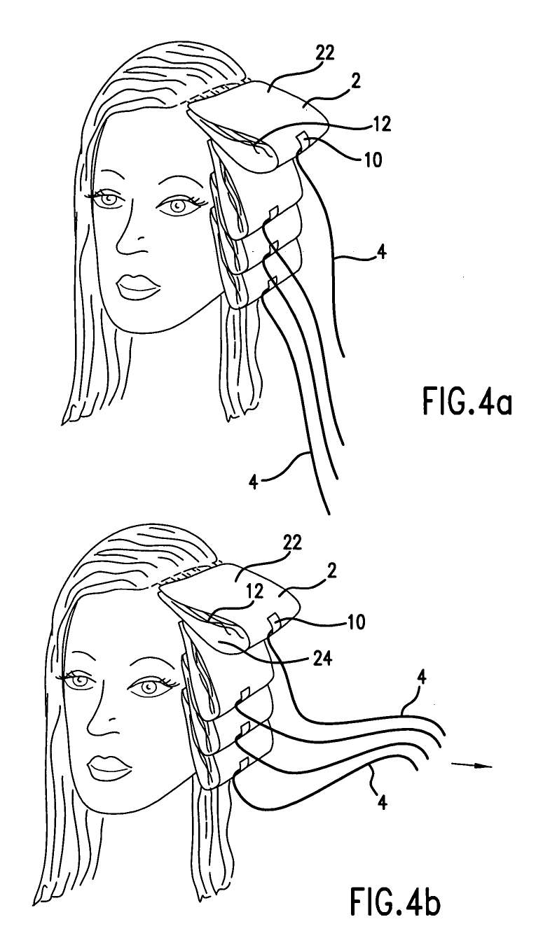

As used herein, the term “interior of the coloring strip” refers to the portion of the coloring strip in which the hair is contained after the coloring strip is folded as in FIG. 3 or FIG. 4. The “exterior of the strip” means the side of the coloring strip that is on a side of the coloring strip that is opposite the interior of the coloring strip, and is external to the hair. Each of the interior and exterior of the coloring strip have a top and a bottom after folding.

FIG. 1 shows the coloring strip having an appendage 4 attached thereto. The coloring strip may be formed of aluminum foil, a plastic sheet, or other sheets of material. A preferred material is a substantially transparent material that resists absorption of the coloring material, such as transparent Mylar. The appendage of FIG. 1 is a string which is attached in substantially the center of the exterior of the coloring strip. The string may be attached by an adhesive, or by an adhesive coated strip 10. The string may be attached by a fastener. By way of example, the coloring strip may be 15 to 35 cm. long, with a width of 7.5 to 12.5 cm, and having a string of 20 to 35 cm. It is preferred that the string have a length that is in excess of the length of the coloring strip.

The coloring strip shown in FIG. 2 has an appendage in the form of a tail that extends from one end of the coloring strip. Again, the coloring strip may be formed of aluminum foil, plastic sheets or other suitable materials. It is preferred that the coloring strip is formed of a transparent material that will not absorb the coloring material. The coloring strip in FIG. 2 is formed as a unitary member, such as by cutting the coloring strip from a larger piece of material, so that the appendage extends from one end of the coloring strip. By way of example, the coloring strip is 15 to 35 cm. long, with a width of 7.5 to 12.5 cm, with an appendage extending therefrom that is 10 to 30 cm inches in length. In the embodiment as shown in FIG. 2, the appendage has substantially the same length as the coloring strip, and after folding, is twice as long as the coloring strip.

FIG. 3a and FIG. 3b show a plurality of coloring strips in position on the client's hair. The coloring strip of FIG. 3a and FIG. 3b is the coloring strip according to the embodiment of FIG. 2. In use, the coloring strip 6 is positioned underneath several strands of hair 12, and the coloring material is coated on the hair that is selected and positioned over the coloring strip. The first end of the coloring strip is positioned underneath the selected hair, and as close to the scalp as possible, so that the coloring material reaches to the roots of the hair follicles. After the coloring material is applied, the coloring strip is folded as shown in FIG. 3a. The coloring strip is folded substantially in half, so that a first end of the coloring strip meets an opposite end of the coloring strip. The selected hair is present within the interior 14 of the coloring strip. The appendage extends to an exterior 16 of the coloring strip, and is of sufficient length to extend beyond, and hang below, the coloring strip as shown in FIG. 3a. When the embodiment of the coloring strip according to the present invention of FIG. 2 is used, one end of the appendage extends from an upper end of the coloring strip when folded, and extends along the upper exterior portion of the coloring strip, so that an opposite end of the appendage extends below the coloring strip.

After the coloring strip is in position for the appropriate time for the coloring materials to sufficiently color the hair, the coloring strips are removed. The coloring strips may be removed by pulling an end of the appendage 8 as shown in FIG. 3b, such as by gripping the appendage between the thumb and the forefinger. The coloring strips may be removed one at a time, and may be removed rapidly because of the ease of removing the coloring strips by pulling on the appendage. Alternatively, several of the strips may be grasped and pulled simultaneously. The coloring strips as shown in FIG. 3a are removed by the upper portion of the folded strip being pulled away from the lower portion, so that the hair of the client is not pulled in a manner that is painful to the client.

FIG. 4a and FIG. 4b show coloring strips according to the embodiment of FIG. 1 in position in the client's hair. Again, strands of hair 12 are selected, and placed over the coloring strip 2, with one end of the coloring strip as close to the scalp as possible. The coloring material is applied, and the coloring strip is folded over the hair, so that a first end of the coloring strip meets an opposite end of the coloring strip, and the hair is retained in an interior 22 of the coloring strip. In this embodiment, the appendage is a string 4 that is attached to, and extends from, an exterior 24 of the coloring strip at approximately the mid-point of the coloring strip, such as by an adhesive strip 10.

After the coloring material has acted upon the hair of the client for an appropriate amount of time, the coloring strips are removed by pulling on the appendage or string as shown in FIG. 4b. The coloring strips may be removed by pulling the appendages away from the client as shown in FIG. 4b, either as a group or one at a time. The appendages of the preferred embodiments allow for rapid removal of the coloring strips, and minimize or avoid exposure of the operator to the coloring material while removing the strips.

It is preferred that the coloring strips are substantially transparent, so that the hair stylist can see the change in the hair coloring through the coloring strips. It is also preferred that the strings of the embodiment in FIG. 1 are of different colors. It is typical in hair coloring to color the hair different shades in different places on the hair. For example, many clients like their hair to be lighter closer to the face, and darker toward the back of the head. The coloring may be varied according to materials used, timing and the like. The use of multiple colored strings allows that hair stylist to know which coloring strips are being used with various coloring materials. Use of multiple colored appendages allow the color coding system to be used with the device.

What is claimed is:

1. A method of hair coloring comprising the steps of:
selecting strands of hair, and positioning said strands of hair over an interior side of a coloring strip;
applying a coloring material to said strands of hair and folding a portion of said coloring strip over a remaining portion of said coloring strip, wherein an appendage that is attached to said coloring strip extends to an exterior side of said coloring strip;
subsequently removing said coloring strip from said strands of hair by pulling on said appendage.

2. A method of hair coloring as described in claim 1, wherein said appendage is a string that extends from said exterior side of said coloring strip.

3. A method of hair coloring as described in claim 1, wherein said appendage extends from an end of said coloring strip to said exterior side of said coloring strip.

4. A method of hair coloring as described in claim 1, wherein said appendage is formed of a material from which said coloring strip is formed.

5. A method of hair coloring as described in claim 4, wherein said coloring strip is formed as a unit.

6. A method of hair coloring as described in claim 4, wherein said coloring strip is formed as a sheet.

7. A hair coloring strip, comprising a foldable sheet, said foldable sheet having an interior side and an exterior side, said hair coloring strip further comprising an appendage, wherein said appendage extends to said exterior side of said hair coloring strip.
